(19) United States
(12) Patent Application Publication
(10) Pub. No.: US 2023/0371879 A1
(43) Pub. Date: Nov. 23, 2023

(54) DEVICE AND METHOD FOR ASSESSING DISCOMFORT AND/OR DISABILITY GLARE OF A SUBJECT

(71) Applicant: Essilor International, Charenton-Le-Pont (FR)

(72) Inventors: Guillaume GIRAUDET, Charenton-Le-Pont (FR); Elisa TARTAGLIA, Charenton-Le-Pont (FR); Anne-Catherine SCHERLEN, Charenton-Le-Pont (FR); Stéphane PERROT, Charenton-Le-Pont (FR); Marie DUBAIL, Charenton-Le-Pont (FR)

(73) Assignee: Essilor International, Charenton-Le-Pont (FR)

(21) Appl. No.: 18/044,708

(22) PCT Filed: Sep. 16, 2021
(86) PCT No.: PCT/EP2021/075538

§ 371 Date: Mar. 9, 2023

(30) Foreign Application Priority Data
Sep. 17, 2020 (EP) 20306041.3

(51) Int. Cl. A61B 5/378; A61B 5/00

(52) U.S. Cl. CPC A61B 5/378; A61B 5/6803

(57) ABSTRACT

The invention relates to a device (10) for assessing discomfort glare and/or disability glare of a subject, comprising: —at least one neuro-sensor (11) for detecting a neural signal linked to the sensitivity of the eyes of the subject, —a control unit (15) adapted to a) record the neural signal of the subject detected by the neuro-sensor while at least one eye of the subject receives a given light condition, b) provide a threshold neural signal that is characteristic of a shift for the subject from no-glare state to discomfort glare and/or disability glare, c) assess whether the neural signal recorded in step a) correlates with a discomfort glare and/or disability glare of the subject by comparing said neural signal recorded in step a) to said threshold neural signal provided in step b). The invention also relates to an eyeglass comprising said device, to a method for assessing discomfort glare and/or disability glare of a subject, and to a virtual reality headset comprising the device.

### TECHNICAL FIELD OF THE INVENTION

The invention relates to the field of assessing discomfort glare and/or disability glare of a subject.

More precisely the invention relates to a device and a method for assessing discomfort and/or disability glare of a subject.

### BACKGROUND INFORMATION AND PRIOR ART

Comfort and visual acuity of a subject may vary depending on the light stimulation which is experienced by this subject. When the light stimulation is too strong, the subject may either experience discomfort glare, that is to say that the subject feels uncomfortable when receiving the light stimulation, and/or disability glare, that is to say that the subject is unable to accurately distinguish a feature in the environment when receiving said light stimulation.

The alteration of the comfort and visual acuity with regard to a light stimulus is specific to each subject and depends on the sensitivity of the eyes of this subject. Therefore, it is important to be able to evaluate the discomfort glare and/or disability glare for each subject in order to determine the appropriate level of light protection necessary for this subject. In other words, the assessment of discomfort glare and/or disability glare will allow an eye care professional to provide this subject with the most appropriate lens, filter, or device that will enable him to feel comfortable in a given light environment.

It is known to assess the discomfort glare and/or disability glare of a subject using subjective methods. In these subjective methods, the subject is either stimulated with a light source and indicates when a discomfort glare is felt, or is stimulated with a light source while asked to look at a feature and indicates when he is unable to distinguish said feature in the environment which signifies that a disability glare is felt. However, these methods are too dependent on the subject's judgment and thus only partially determine the discomfort glare and/or disability glare of the subject.

### SUMMARY OF THE INVENTION

Therefore one object of the invention is to provide a device for assessing discomfort glare and/or disability glare of a subject, objectively and accurately.

The above object is achieved according to the invention by providing a device for assessing discomfort glare and/or disability glare of a subject, comprising:

at least one neuro-sensor for detecting a neural signal linked to the sensitivity of the eyes of the subject,

a control unit adapted to

a) record the neural signal of the subject detected by the neuro-sensor while at least one eye of the subject receives a given light condition,

b) provide a threshold neural signal that is characteristic of a shift for the subject from a no-glare state to discomfort glare and/or disability glare, and,

c) assess whether the neural signal recorded in step a) correlates with a discomfort glare and/or disability glare of the subject by comparing said neural signal recorded in step a) to said threshold neural signal provided in step b).

Thus, the device is able to record a neural signal of a subject while the subject is receiving a given light condition, and to analyze the signal by comparison to a threshold signal. Depending on the result of this comparison, the device objectively assesses whether, under said given light condition, the subject experiences discomfort glare and/or disability glare.

The device does not require the subject to answer questions relative to his discomfort and/or disability when he receives the given light condition. On the contrary, the subject may only focus on the light condition he receives or on a feature he is asked to look at. The analysis of the neural signal guarantees that the assessment of discomfort glare and/or disability glare is objective.

According to an embodiment of said device, the neural signal linked to the sensitivity of the eyes originates from at least one specific area of the brain.

For instance, the neural signal originates from the prefrontal area of the brain, and/or from the occipital area of the brain.

Therefore, the neural signal is easily accessible.

In particular, in that embodiment of said device, in step c), the control unit is adapted to:

determine at least one specific feature of the recorded neural signal originating from said at least one specific area of the brain of the subject,

compare said at least one specific feature of the recorded neural signal to a threshold specific feature of the threshold neural signal originating from the same at least one specific area of the brain of the subject, and

depending on the result of said comparison, assess whether the subject is experiencing discomfort glare and/or disability glare under said given light condition.

Therefore, the control unit compares the recorded neural signal and the threshold neural signal by comparing specific features extracted from said signals, and concludes whether the subject is experiencing discomfort glare and/or disability glare based on this comparison.

Other characteristics of the device of this invention, taken together or separately, are the following:

the specific feature of the neural signals is at least one of the specific features chosen from the following list: a time of recovery of the stimulated neurons in said at least one specific area of the brain of the subject, or an amplitude of the neural signal originating from said at least one area of the brain of the subject at a chosen time after said at least one eye of the subject received said given light condition;

in step b), the threshold neural signal originates from a prefrontal area of the brain and exhibits a threshold time of recovery of the stimulated neurons that is comprised between 300 milliseconds and 500 milliseconds, preferably between 350 ms and 450 ms;

the neuro-sensor is further able to detect the neural signal originating from another area of the brain of the subject;

the specific feature of the neural signals that is determined by the control unit is the ratio between a maximum amplitude of a peak in the neural signal originating from said at least one area of the brain and a maximum amplitude of a peak in the neural signal originating from said other area of the brain;

the threshold ratio between the maximum amplitude of the peak in the threshold neural signal originating from a prefrontal area of the brain and the maximum amplitude of the peak in the threshold neural signal originating from an occipital area of the brain is comprised between 0.8 and 1.2;

the device further comprises at least one light source that is operated by the control unit to expose said at least one eye of the subject to said given light condition;

the control unit is adapted to operate said light source to expose said at least one eye of the subject to a plurality of given light conditions at a plurality of chosen times, and wherein in step b), the control unit is furthermore adapted to:
record a plurality of neural signals originating from said at least one area of the brain of the subject in response to the exposure of said at least one eye of the subject to each chosen light conditions, and,
compare with each other the recorded neural signals to evaluate a change in brain activity as a function of the light condition received by said at least one eye,
determine the threshold neural signal as the recorded neural signal for which the change in brain activity occurs;

the change in brain activity is labelled at least by one of the following measures: a lengthening in the time of recovery of the neurons in the neural signals originating from a same area of the brain of the subject, a decrease in the amplitude of the neural signals originating from a same area of the brain of the subject at a chosen time after said at least one eye of the subject received the light condition, or a shift in the ratio of maximum amplitude between the neural signals originating from said at least one area of the brain of the subject and the neural signals originating from another area of the brain activity;

there is a discomfort threshold neural signal defining the shift from the no-glare state to discomfort glare, a pain threshold neural signal defining the shift from discomfort glare to painful glare and a disability threshold neural signal defining the shift from the no-glare state to disability glare;

the given light condition received by said at least one eye of the subject, is defined by a spectral parameter, a spatial parameter, a temporal parameter and an intensity parameter;

only one of said spatial, spectral, temporal and intensity parameters of the light condition varies in the plurality of given light conditions to which is exposed the at least one eye of the subject, all three other of said parameters being fixed;

the device further comprises a screen displaying an image to said at least one eye of the subject;

the neuro-sensor comprises a plurality of electrodes placed on the head of the subject, at least in one the following regions: in a forehead region above the eyes of the subject, or in a back region behind the head of the subject;

in step a), the eye of the subject is provided with a filter through which the light passes before reaching the at least one eye of the subject.

In another embodiment of the device, the neural signal linked to the sensitivity of the eyes originates from the retina of the subject.

A further object of the invention is to provide an eyeglass comprising:
the device of the invention and
an active filter defined at least by one variable parameter chosen among a transmission value and/or a spectrum range,

wherein the control unit is adapted to determine a value for said variable parameter of the active filter based on the assessing of the discomfort glare and/or disability glare implemented by said device when the subject receives the light condition through said eyeglass.

A further object of the invention is to provide a method for assessing discomfort glare and/or disability glare of a subject, objectively.

More precisely, the above object is achieved according to the invention by providing a method for assessing discomfort glare and/or disability glare of a subject, comprising the following steps:

a) recording a neural signal linked to the sensitivity of the eyes of the subject while at least one eye of the subject receives a given light condition,

b) providing a threshold neural signal that is characteristic of a shift for the subject from a no-glare state to discomfort glare and/or disability glare,

c) assessing whether the neural signal recorded in step a) correlates with a discomfort glare and/or disability glare of the subject by comparing said neural signal recorded in step a) to said threshold neural signal provided in step b).

Therefore, in the method of the invention, the subject does not necessarily have to answer a question in order to assess his visual discomfort and/or disability relative to the given light condition he receives. As it will be explained further in the description, in order to establish the threshold neural signal provided in step b), the subject may however be asked to answer a question relative to his visual discomfort and/or disability when receiving a given light condition.

On the contrary, in an embodiment of the method, in step a), the subject is silent and still, in a silent environment.

Therefore the recorded neural signal is directly linked to the light received by the eye(s) of the subject and to the sensitivity of the eyes as regards said received light.

In an embodiment of the method, the neural signal is recorded in at least one area of the brain of the subject. Therefore, the neural signal is easily accessible.

Another object of the invention is to provide a method for determining a parameter that is characteristic of a light filter to be provided to a subject in order to maintain or improve the visual comfort and/or visual acuity of said subject for a given light condition, comprising the following steps:

determining a threshold light condition characteristic of a shift for the subject from no-glare state to discomfort glare and/or disability glare,

determining for each light condition among a group of light conditions, an index representative of the level of protection required by the subject, based on said light condition threshold,

determining a score for each light condition among the group of light conditions and for each filter among a group of filters, said score being representative of the capacity of the filter to reach the level of protection required by the subject, based on said index, and

determining at least one filter among the group of filters based on the scores of said at least one filter in a plurality of light conditions among the group of light conditions,

wherein the determination of the threshold light condition is implemented by the device of the invention wherein the control unit:
operates said light source to expose said at least one eye of the subject to a plurality of given light conditions at a plurality of chosen times, and
determines that the threshold light condition is the light condition for which the ratio of maximum amplitude values between the neural signals originating from said at least one area of the brain of the subject and the neural signals originating from another area of the brain activity is the closest to a threshold ratio characteristic of a shift for the subject from no-glare state to discomfort glare and/or disability glare.

According to an embodiment of this method, the parameter that is characteristic of the filter is at least one of the following parameters: the transmission value, the spectrum range and the dynamic laws of variation of the transmission value and/or of the spectrum range.

According to an embodiment of this method, the control unit determines a law of variation of the transmission value and/or of the spectrum range of the filter based on the light condition received by said filter, said method being implemented with the device of the invention, and wherein the law is determined as follows: for each light condition sent to the eye of the subject and that leads to a ratio strictly greater than the threshold ratio, the transmission value of the filter is decreased and/or the spectrum range is modified in order to force said ratio to be equal to the threshold ratio.

According to an embodiment of this method, the filter is an active filter, such as an electrochromic system or a photochromic system, that exhibits a variable parameter, such as a variable transmission value or a variable spectrum range, configured with said law of variation.

According to an embodiment of this method, the filter exhibits a fixed tint, said tint being selected based on said law of variation and depending on the light conditions wherein the subject is the most likely to wear said filter.

Another object of the invention is to provide a virtual reality headset intended to be worn by a subject and that includes the device here-above described. More precisely, the virtual reality headset comprises:
a device as described above; and,
a fastening unit for keeping said device in front of the eyes of said subject.

The headset may eventually comprise an isolating unit for isolating said subject from ambient light.

### DETAILED DESCRIPTION OF EXAMPLE(S)

The following description with reference to the accompanying drawings will make it clear what the invention consists of and how it can be achieved. The invention is not limited to the embodiment/s illustrated in the drawings. Accordingly, it should be understood that where features mentioned in the claims are followed by reference signs, such signs are included solely for the purpose of enhancing the intelligibility of the claims and are in no way limiting on the scope of the claims.

In the accompanying drawings:

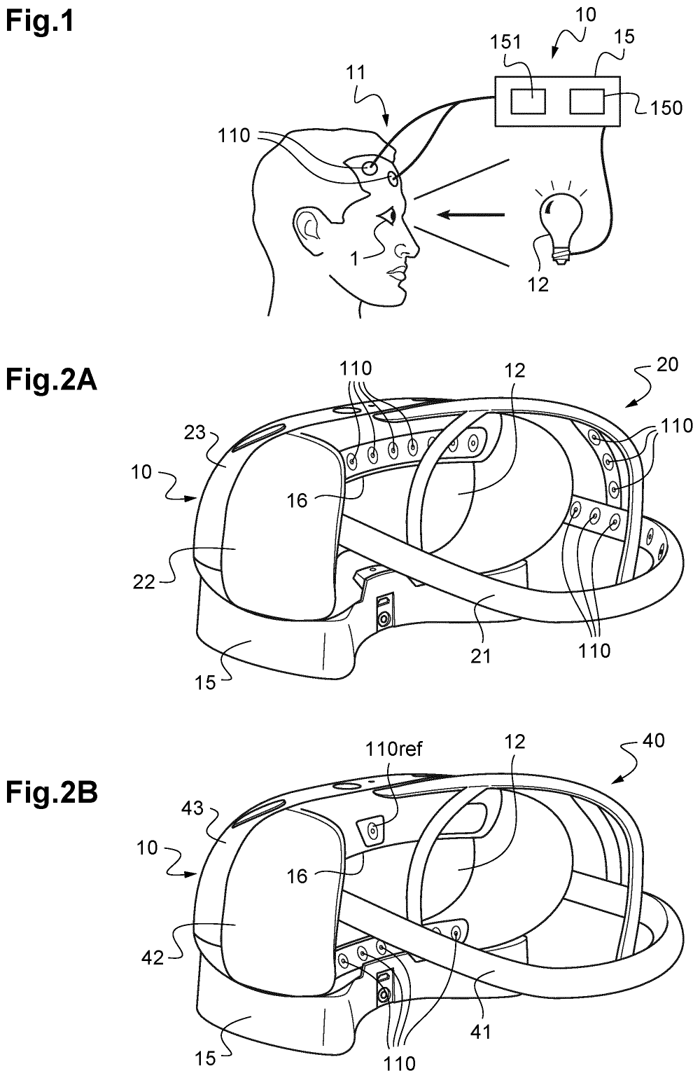

FIG. 1 is a schematic view of an embodiment of the device according to the invention;

FIG. 2A is a schematic view of a first embodiment of the virtual reality headset according to the invention;

FIG. 2B is a schematic view of a second embodiment of the virtual reality headset according to the invention;

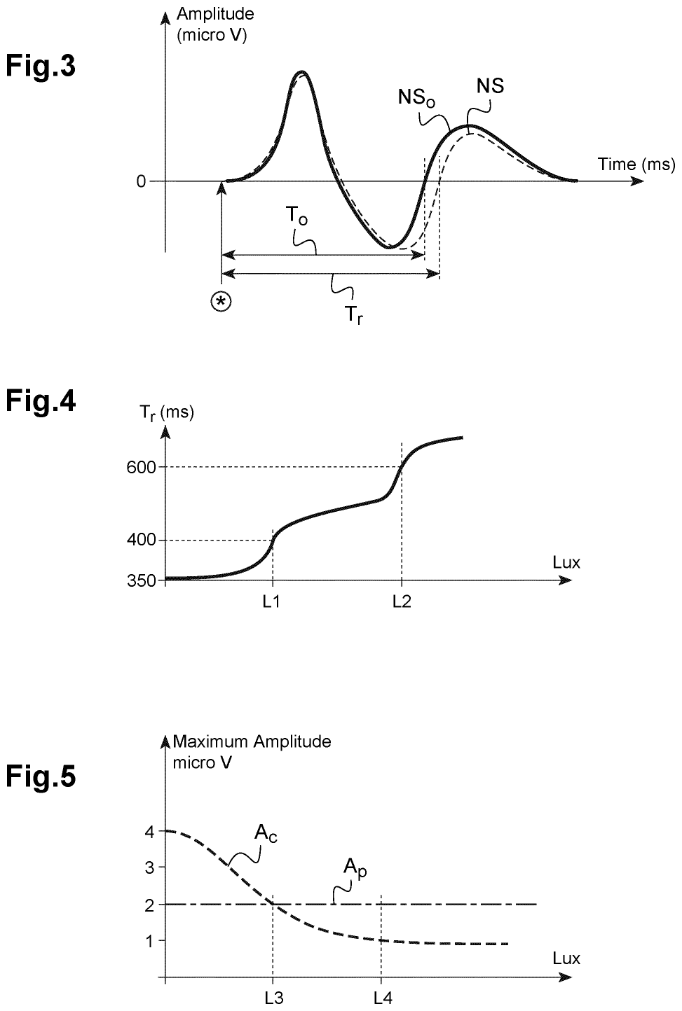

FIG. 3 is a schematic representation of an example of a recorded neural signal in the prefrontal area of the brain of a subject, the ordinate axis (y-axis) giving the amplitude of the signal (in microvolts) and the abscissa axis (x-axis) giving the time (in milliseconds (ms));

FIG. 4 is an example of a graph representing the time of recovery of the stimulated neurons in the prefrontal area of the brain of the subject (in milliseconds ms), as a function of the light intensity (in Lux) received by the eye(s) of the subject;

FIG. 5 is an example of a graph representing a first curve Ap that is the maximum amplitude of neural signals originating from the prefrontal area of the brain of the subject as a function of the light intensity (in Lux) received by the eye(s) of the subject and a second curve Ac that is the maximum amplitude of neural signals originating from the occipital area of the brain of the subject as a function of the light intensity (in Lux) received by the eye(s) of the subject; and,

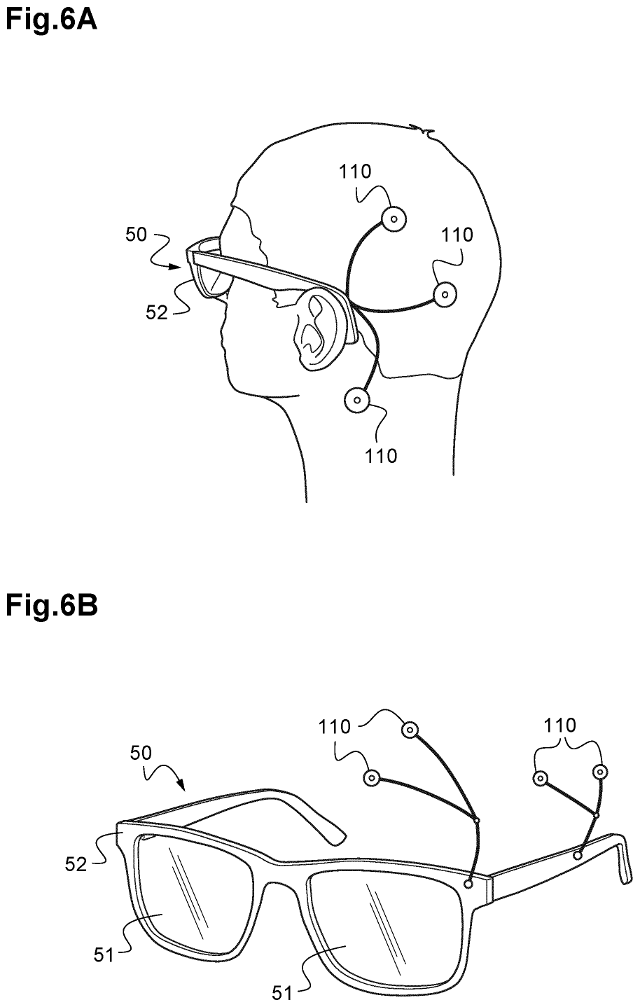

FIGS. 6A and 6B are two examples of an eyeglass that includes the device of the invention.

The present invention provides a device 10 and a method for objectively assessing discomfort glare and/or disability glare of a subject. The device 10 and the method of the invention are objective diagnosis tools.

As explained in the introduction, the discomfort glare is an alteration of the visual comfort experienced by the subject when at least one eye of the subject receives a given light condition, and disability glare is an alteration of the visual performance of the subject when said at least one eye of the subject receives a given light condition. Discomfort and/or disability glare are thus any relatively intense and prolonged reaction or modification of visual comfort or visual performance of a subject in relation to a given light condition received by the eye.

In other words, discomfort glare corresponds to a state of the subject wherein said subject experiences an uncomfortable feeling because of the light condition the subject receives in one or both eyes. Discomfort glare can even turn into painful glare which is a state of the subject wherein said subject experiences not only discomfort but even pain because of the light condition the subject receives in one or both eyes. Disability glare corresponds to a state of the subject wherein said subject experiences an inability to accurately distinguish a feature in the environment, such as an image, an object, a word, or a letter, because of the light condition the subject receives in one or both eyes.

On the contrary, the no-glare state is a state of the subject wherein said subject is not experiencing any glare. In the no-glare state, the given light condition that the subject receives in one or both eyes does not induce a visual discomfort or a visual disability for the subject.

Of course, discomfort glare, painful glare, and disability glare are associated with a given light condition received by the eye of the subject, but they are not necessarily reached for a same given light condition, for a given subject. Indeed, discomfort glare, painful glare and disability glare are linked to the sensitivity to light of the eyes of the subject. And discomfort glare, painful glare, and disability glare are personal, and can therefore be experienced for different light conditions from one subject to another.

If it appears that said given light condition yields to discomfort glare and/or disability glare, it is then possible to provide the subject appropriate tools (such as lens filters) that will make him feel more comfortable or experience better visual performance even when his eyes receive this given light condition. The device 10 and the method of the invention make it easier for an eye care professional (optician, optometrist, ophthalmologist, etc) to determine which light condition makes a subject feel uncomfortable or visually disable and therefore to determine which kind of protection may improve the visual comfort and/or visual performance of this subject under certain light condition or under certain light environments. This application will be further detailed at the end of the description.

To know whether the subject experiences discomfort glare and/or disability glare, under a given light condition, the device 10 and/or the method of the invention measure physical responses of the subject when one or both of his eyes receives said given light condition.

More precisely, the device 10 and the method of the invention objectively assess the discomfort glare and/or disability glare of a subject, based on the analysis of a neural signal of the subject that is recorded while said subject receives a given light condition in at least one of his eyes 1.

Device

As shown on FIG. 1, the device 10 of the invention comprises:

at least one neuro-sensor 11 for detecting a neural signal linked to the sensitivity of the eyes 1 of the subject, and

a control unit 15 adapted to

a) record the neural signal of the subject detected by the neuro-sensor while at least one eye 1 of the subject receives a given light condition,

b) provide a threshold neural signal that is characteristic of a shift for the subject from a no-glare state to discomfort glare and/or disability glare,

c) assess whether the neural signal recorded in step a) correlates with a discomfort glare and/or disability glare of the subject by comparing said neural signal recorded in step a) to said threshold neural signal provided in step b).

The neuro-sensor 11 of the device 10 may comprise at least one electrode 110, preferably a plurality of electrodes 110.

The neuro-sensor 11 may be adapted to record the neural signal originating from at least one area of the brain of the subject. Such neural signals may be electroencephalograms.

In that aim, the electrodes 110 of the neuro-sensor 11 may be placed on the head of the subject. The electrodes 110 are able to detect the electrical activity of specific neurons that are stimulated when the eyes receive information, here light information. The neural signal detected by these electrodes 110 is linked to the sensitivity of the eyes because such neural signal is that of said specific neurons. The neural signal detected by such electrodes should exclude all the electrical activity that could be induced by muscular activity of the eye, that would for instance be linked to an eyelid movement. In other words, the neural signal of interest in the present invention focuses only on the sensory information sent to the brain of the subject, through the optic nerve, by the photoreceptor of the eye(s) receiving the light condition. The electrical activity that is due to muscular activity of the eyes is called a “muscular signal”. The muscular signal possibly mixed with the neural signal is considered to be a noise detected by the electrodes and should be removed from the neural signal. In practice, the muscular signal generally exhibits an electrical activity of several millivolts (mV), whereas the neural signal on which the invention focuses exhibits an electrical activity of a few microvolts (μV or micro V) only. Moreover, in the frequency domain, the frequencies exhibited by a muscular signal are generally comprised between 0.1 and 0.5 Hertz (Hz), whereas the frequencies exhibited by a neural signal according to the invention are generally around 5 to 10 Hz. Such distinctive features help removing the muscular signal from the neural signal, in cases both are detected by the electrodes.

The electrodes 110 are here able to detect the neural signal that originates from the neurons of the prefrontal area of the brain of the subject. For instance, these electrodes 110 are placed in a forehead region of the head of the subject, above the eyebrows of the subject.

Here, the example of neuro-sensor 11 that is represented on the device 10 of FIG. 1 comprises a plurality of electrodes 110, that are able to record electroencephalograms originating from the prefrontal and/or occipital areas of the brain of a subject.

As shown on FIG. 3, the neural signal recorded by the electrodes 110 is here the signal that gives the electrical activity of the neurons as a function of time (that is to say in the discrete time domain). The electrical activity of the neurons varies over time in response to the light condition received by the eye(s) of the subject. On FIG. 3, the arrow and the asterisk symbolize the instant at which the light condition is sent to the eye(s) 1. In practice, it is considered that the instant when the light condition is sent to the eye 1 is identical to the instant when the eye 1 receives said light condition. The fact that the light condition is sent to or received by the eye 1 is commonly called “stimulation” or “light stimulation”.

The recording of the neural signal by the neuro-sensor 11 is preferably performed shortly before, during and following the light stimulus provided by at least one light source, in order to correlate the recorded neural signal to the light condition received by the eye 1 of the subject.

The neural signal recorded for assessing the discomfort glare of a subject may be different from the neural signal recorded for assessing the disability glare of a subject, in particular as regards its shape. Indeed, for assessing the disability glare, the subject is asked to perform a visual task, in addition to receiving the light condition in his eyes, and said visual task is likely to impact the neural signal. Moreover, the neural signal recorded for assessing the discomfort glare might originate from a different area of the brain that the neural signal recorded for assessing the disability glare.

In the present invention, the subject can have in front of one or both of his eyes 1 a filter or corrective lenses for instance. This does not affect the invention and its operation. For instance, according to an embodiment of the device of the invention, in step a), the eye of the subject is provided with a light filter through which the light passes before reaching the at least one eye of the subject. In other words, the neural signal of the subject is recorded for a light condition that has been modified by the light filter before it is received by the eye of the subject.

The given light condition received by said at least one eye of the subject is defined by a spectral parameter, a spatial parameter, a temporal parameter and an intensity parameter.

The spectral parameter of the light condition may be the wavelength of the light that reaches the eye or the range of wavelengths that composes the light reaching the eye. For instance, when the light is white visible light, it comprises a plurality of wavelengths, preferably the whole continuous spectrum within the range 380 nanometers (nm) −780 nanometers (nm). The spectral parameter may therefore be the whole range [380 nm; 780 nm] (white light). On the contrary, when the light received by the eye is a colored light, such as red light, the spectral parameter may be the corresponding wavelength or range of wavelength of said red light, for instance the wavelength 700 nm, or the whole range [650 nm; 750 nm].

The spatial parameter of the light condition defines the homogeneity or inhomogeneity of the light in space. For instance, the light received by the eye could be homogeneous, which implies that the eye 1 receives the same light condition from every direction in the environment the subject is looking at. A spatial parameter homogeneous is for instance that of a diffused light source. A spatial parameter homogeneous is for instance that of a very wide light source. On the contrary, the spatial parameter of the light received by the eye 1 could be heterogeneous, that is to say that depending on the direction under which the light enters the eye 1, said light exhibits different spectral and/or temporal and or intensity parameters. A spatial parameter heterogeneous is that of a punctual light source for instance. In the present case, the light condition is preferably spatially homogeneous. In other words, the light condition has no contrast so that its Michaelson contrast value is equal to 0.

The temporal parameter of the light condition may be the duration over which the eye 1 receives the light. In other words, the temporal parameter may be the continuous or temporarily aspect of the light received by the eye 1. For instance, if the temporal parameter of the light may be a few milliseconds, such as less than 1 second, it implies that the eye 1 receives the light in the form of a flash or impulse. On the contrary, if the temporal parameter of the light is several tens of seconds or up to one minute, the eye 1 receives the light in the form continuous illumination. In the present case, the light condition is preferably a flash.

The intensity parameter refers to the light flux emitted by the light source, given in Lumen, or to the illuminance received by the eye(s) of the subject, expressed in Lux. Notably, the intensity parameter of the light condition could be the illuminance that gives the light flux per square meters of illuminated surface, given in lux, for example the illuminance could be comprised between 20 lux to 10000 lux. In alternative, the intensity parameter of the light condition may be the luminance expressed in candelas per square meter. In the present case, the luminance could be comprised between 20 candelas per square meter (cd/m2) and 8000 cd/m2. The luminance is the luminous intensity per unit area of light travelling in a given direction. In a further alternative, the intensity parameter of the light condition could take into account the diameter of the pupil of the eye of the subject, and would therefore be given in troland. To convert the luminance in troland, it is necessary to multiply the area of the pupil of the subject by the luminance of the light source. In this further alternative, the intensity parameter of the light condition received by the eye of the subject could therefore be standardized so that, whatever the diameter of the pupil of the subject, two subjects may receive the same amount of light in their eyes. In case the intensity parameter is given in troland, the device 10 may comprise a sensor, such as a camera, to detect and measure the size of the pupil of the subject receiving the light condition. In the present case, the light condition preferably has an intensity parameter that is constant over the duration of the flash of homogeneous light sent to the eye of the subject.

It is to be understood that each of said parameters defining the light condition may impact the discomfort glare and/or disability glare of the subject, depending on the sensitivity of the eyes to each parameter.

In the following example of the description, the subject preferably receives a same light condition in both eyes 1, simultaneously. The light condition is a flash of homogeneous light, of about 600 ms, comprising the wavelengths ranging from 380 nm to 780 nm, and the intensity parameter of the light condition is comprised between 20 lux and 10000 lux.

It is to be noted that in the present description, the “glare” is preferably diagnosed (or assessed) in relation with the intensity of the homogeneous light that is received by the eye and not in relation with a possible spatial contrast.

The device 10 here may comprise at least one light source 12 that is operated by the control unit 15 to expose said at least one eye of the subject to said given light condition. The device represented on FIG. 1 here includes such light source.

Said at least one light source 12 is preferably combined with a diffuser (not represented) disposed in front of the user's eyes to provide a diffused light, that is to say a homogeneous light. In this case, the light source 12 emits light toward the diffuser, which itself emits light toward the eyes of the subject. Alternatively or in combination, the light source 12 may be positioned to emit light directly toward one or both eyes of the user. Hence, the device 10 may be configured to expose the user to either a homogeneous or punctual light, or both simultaneously. Preferably, in the present case, the device 10 is configured to expose the eye to homogeneous light. It means that there is no spatial contrast of the light that is seen by the subject. Suh a spatial contrast, that is not seen here, would for example be that of a pattern showing small points of light of different brightness (described in terms of Michaelson contrast value).

Light source 12 preferably comprises at least one light-emitting diode (LED) able to have variable light spectrum as RGB LEDs (Red-Green-Blue light emitting diodes) or RGB-W LEDs (Red-Green-Blue-White light emitting diodes). Alternatively, said light source 12 may be configured to provide a predetermined single white light spectrum or, alternatively, a spectrum having all visible radiations with substantially the same intensity, in contrast with a spectrum having peaks. Said at least one light source 14 is preferably controlled with a constant current to obtain a constant light flux coming out said at least one light source 14. Providing the user with a constant light flux allows reducing or avoiding biological effects disturbances compared to light sources controlled with Pulse Width Modulation (PWM).

In alternative, the control unit of the device could operate a light source that is not comprised within the device. In alternative again, the light source could be distinct from the device and operated manually, for instance by an operator.

In addition, the device 10 can also comprise a screen (not represented) displaying an image (or a feature) to said at least one eye of the subject. This screen is used when the disability glare of the subject is assessed. In that case, the eye of the subject both receives the light condition and looks at the image displayed on the screen while the neural signal is recorded.

The control unit 15 is configured to communicate with the neuro-sensor 11, and, if appropriate with the light source 12 and the eventual screen. This communication may be established either with wireless communication means or with line-based communication means.

The control unit 15 may be at distance from the subject. In an alternative embodiment it could be worn by the subject. In the example shown on FIGS. 1 to 5, the control unit 15 is placed at distance from the subject. In the examples shown on FIGS. 6A and 6B the control unit 15 is worn by the subject.

As shown on FIG. 1, the control unit 15 may comprise a memory 150 and a processor 151 that communicate with one another and with said neuro-sensor 11 and light source 12. The control unit 15 is for instance integrated in a computer.

In particular, the memory 150 may be configured to receive the recorded neural signal from the neuro-sensor 11 and to store it in order for the processor 151 to analyze said recorded neural signal and assess the glare state of the subject due to the light condition received by the eye when the neural signal was recorded.

The memory 150 may be also configured to store the threshold neural signal in order for the processor 151 to analyze it and compare the recorded and threshold neural signals to one another.

The threshold neural signal may be a neural signal originating from the same area of the brain as the recorded neural signal to which it is compared. In the present example, the threshold neural signal originates from the same area of the brain as the recorded neural signal to which it is compared.

The threshold neural signal is a typical neural signal that characterizes the shift from a no-glare state to discomfort glare and/or disability glare of a subject.

There is a discomfort threshold neural signal defining the shift from the no-glare state to discomfort glare, a pain threshold neural signal defining the shift from discomfort glare to painful glare, and a disability threshold neural signal defining the shift from the no-glare state to disability glare.

All three threshold neural signal could be identical or distinct and different. In the present example, it is considered that the discomfort, pain and disability threshold neural signals are distinct and different. Notably, the discomfort threshold neural signal and pain threshold neural signal may originate from a different area of the brain of the subject than the disability threshold neural signal. Moreover, the shape of the discomfort threshold neural signal and pain threshold neural signal might be different from the shape of the disability threshold neural signal.

Each threshold neural signal may either be an individual threshold neural signal that has been determined specifically for the subject undergoing the diagnosis, or a mean threshold neural signal that has been established based on the sensitivity to light of a group of individuals. In any case, each threshold neural signal may be known from the device 10. In the present example, it is considered that each threshold neural signal is known and stored in the memory 150 of the control unit 15 of the device 10.

The control unit 15, and more particularly the processor 151 of the control unit 15, may be configured to execute, in step c), the following actions:

determine at least one specific feature of the recorded neural signal originating from said at least one specific area of the brain of the subject,

compare said at least one specific feature of the recorded neural signal to a threshold specific feature of the threshold neural signal originating from the same at least one specific area of the brain of the subject, and

depending on the result of said comparison, assess whether the subject is experiencing discomfort glare and/or disability glare under said given light condition.

To determine the specific feature of the recorded neural signal and the threshold specific feature of the threshold neural signal, the processor 151 may either analyze the neural signal in the discrete time domain, or apply a fast Fourier transform to transform said discrete time domain data in the frequency domain.

The processor 151 of the control unit 15 may be configured to extract the specific features from the recorded and threshold neural signals and to directly compare said specific features in order to conclude on the glare state of the subject, under the light condition at which the neural signal was recorded.

The control unit 15 therefore objectively concludes on the glare state of the subject, that is to say on the fact that the subject is either not experiencing any glare, or is experiencing visual discomfort and/or visual disability without needing the subject to give his subjective feeling, but directly based on the comparison of specific features.

The rest of the description of the device 10 mainly corresponds to the assessment of glare discomfort. It will be explained when necessary, the differences when the device 10 is used to assess glare disability, which principle is very similar to the assessment of glare discomfort.

The specific feature of the neural signal may be a feature of the neural signal that shows how the neurons of the subject recover after they are stimulated by the light condition received in the eye of the subject. The recovery of the neurons is here defined as the return to a baseline electric activity from which the neurons can be stimulated again.

The specific feature of the neural signal may be at least one of the specific features chosen from the following list: a time of recovery of the stimulated neurons in said at least one specific area of the brain of the subject, or an amplitude value of the neural signal originating from said at least one area of the brain of the subject at a chosen time after said at least one eye of the subject received said given light condition.

More precisely, as shown on the example of FIG. 3, when the eye of the subject is stimulated with a given light condition (assessment of glare discomfort), the recorded neural signal NS originating from the prefrontal area of the brain of the subject comprises two positive peaks interrupted by a negative peak. The first positive peak is linked to the activation of the neurons in that area of the brain due to the light stimulation received in the eye 1 of the subject, and the second positive peak that exhibits a maximum amplitude value smaller than that of the first positive peak is linked to the recovery of the neurons after their activation. The discomfort threshold neural signal NS₀ defining the shift from the no-glare state to discomfort glare, originating from the prefrontal area of the brain, exhibits a similar aspect as the recorded neural signal NS.

The time of recovery Tr, T₀ of the neurons is here defined as the time that has flown between the instant when the light condition is sent to the eye 1 and the instant when the amplitude of the neural signal Ns, NS₀ becomes positive again, in between the two positive peaks.

The processor 151 of the control unit 15 may be configured to compare the time of recovery Tr that can be read on the recorded neural signal NS and the threshold time of recovery To that can be read on the threshold neural signal NS₀.

If the time of recovery Tr of the recorded neural signal NS is greater than the threshold time of recovery To of the threshold neural signal NS₀, then the control unit 15 is configured to conclude that the subject is experiencing discomfort glare (and/or disability glare). On the contrary, if the time of recovery Tr of the recorded neural signal NS is smaller than or equal to the threshold time of recovery T₀, then the control unit 15 is configured to conclude that the subject is not experiencing any glare, which is equivalent to saying that the subject is in the no-glare state.

In this example, for a discomfort threshold neural signal NS₀ defining the shift from the no-glare state to discomfort glare, originating from the prefrontal area of the brain, the threshold time of recovery T₀ of the stimulated neurons, as defined here above, is comprised between 300 milliseconds (ms) and 500 milliseconds (ms), preferably between 350 ms and 450 ms. For a threshold neural signal defining the shift from discomfort glare to painful glare, originating from the prefrontal area of the brain, the threshold time of recovery of the stimulated neurons, as defined here above, is comprised between 500 ms and 700 ms, preferably between 550 ms and 650 ms.

In alternative, the time of recovery of the neurons could be defined as the time that has flown between the instant that corresponds to the maximum amplitude value of the first positive peak in the neural signal originating from the prefrontal area of the brain, and the instant when the amplitude of said neural signal becomes positive again, in between the two positive peaks.

Another way of comparing the neural signals is to compare the amplitude value of both the recorded and the threshold neural signals NS, NS₀ at a chosen instant after said at least one eye 1 of the subject received said given light condition.

Preferably, the chosen instant for comparing the amplitude values of the recorded and threshold neural signals NS, NS₀ is an instant chosen in between the two positive peaks, when the amplitude starts to increase again after the minimum amplitude value of the negative peak was reached.

If the amplitude value, at said chosen time, of the recorded neural signal NS, is smaller than the threshold amplitude value at said chosen time of the threshold neural signal NS₀, then the control unit 15 is configured to conclude that the subject is experiencing discomfort glare and/or disability glare. On the contrary, if the amplitude value, at said chosen time, of the recorded neural signal NS, is greater than or equal to the threshold amplitude value at said chosen time, then the control unit 15 is configured to conclude that the subject is not experiencing any glare (the subject is in the no-glare state). Of course, this reasoning applies when the amplitude values are compared and not the amplitude absolute values.

In an alternative embodiment (not shown), the neuro-sensor is not only able to detect the neural signal originating from one area of the brain of the subject, but further able to detect the neural signal originating from another area of the brain of the subject. In that aim, the electrodes of the neuro-sensor are placed on the head of the subject, in two distinct regions of the head. The electrodes are able to detect the electrical activity of specific neurons that are only stimulated when the eyes receive information (here light information), said specific neurons being located in both areas of the brain. The neural signals detected by these electrodes are linked to the sensitivity of the eyes because such neural signals are that of said specific neurons.

More precisely, the electrodes are here placed in a forehead region of the head of the subject, above the eyebrows of the subject, and in a back region behind the head of the subject. The electrodes in the forehead region are able to detect the neural signal that originates from the prefrontal area of the brain of the subject, and the electrodes in the back region to detect the neural signal that originates from the cortical area of the brain of the subject.

The neuro-sensor may therefore be configured to simultaneously record two neural signals, one originating from each area of the brain.

The memory of the control unit may be configured to store a threshold neural signal originating from the prefrontal area and another threshold neural signal originating from the cortical area of the brain.

As explained before, the processor of the control unit may be configured to determine a specific feature from the recorded neural signals, and to compare said specific feature to a threshold specific feature of the threshold neural signals in order to conclude on the glare state of the subject, due to the light condition at which the neural recorded were recorded.

In that case, the specific feature is linked to the neural signals originating from both areas of the brain. Here, the specific feature of the neural signals shows a comparative activity of the neurons in the two areas of the brain of the subject, which indicates which area is the most active. More precisely, the specific feature is here the ratio of a maximum (positive) amplitude value of the neural signal originating from the prefrontal area of the brain over a maximum (positive) amplitude value of the neural signal originating from the cortical area of the brain.

The processor of the control unit is configured to calculate said ratio by extracting the maximum amplitude values from each recorded neural signals and to calculate the threshold ratio by extracting the maximum amplitude values from each threshold neural signals.

The maximum amplitude value is for instance the maximum amplitude value of a precise peak in the neural signal originating from the prefrontal area and the maximum amplitude value of another precise peak of the neural signal originating from the cortical area.

The processor of the control unit is then configured to compare the ratio that is calculated from the recorded neural signals and the corresponding threshold ratio that is calculated from the threshold neural signals.

If the ratio between said maximum amplitude values is greater than the threshold ratio calculated from the threshold neural signals, then the control unit 15 is configured to conclude that the subject is experiencing discomfort glare (and/or disability glare). On the contrary, if the ratio calculated from the recorded neural signals is smaller than or equal to the threshold ratio, then the control unit 15 is configured to conclude that the subject is not experiencing any glare (or is in the no-glare state).

In this example, for discomfort threshold neural signals defining the shift from the no-glare state to discomfort glare, the threshold ratio of the maximum amplitude value in the threshold neural signal originating from the prefrontal area of the brain over the maximum amplitude value of the threshold neural signal originating from the occipital area of the brain is comprised between 0.8 and 1.2. For pain threshold neural signals defining the shift from discomfort glare to painful glare, the threshold ratio between said maximum amplitude values is comprised between 1.8 and 2.2.

In a further aspect of the invention, the device 10 is configured to proceed to the determination of the threshold neural signals, and/or the threshold specific features.

To do so, the control unit 15 is configured to operate said light source 12 to expose said at least one eye 1 of the subject to a plurality of given light conditions at a plurality of chosen times. Each light condition is sent to the at least one eye 1 at a different time, so that the eye 1 receives successively the plurality of light conditions.

Preferably, only one of said spatial, spectral, temporal and intensity parameters of the light condition varies in the plurality of given light conditions to which is exposed the at least one eye 1 of the subject, all three other of said parameters being fixed.

Here, only the intensity parameter of the light condition varies. Hence, the light condition is a flash (temporal parameter) of homogenous (spatial parameter) and white light (spectral parameter). Only the luminance (intensity parameter) of the light condition varies from around 20 lux to around 10000 lux. Preferably, the eye 1 of the subject receives less intense light at first, and successively more and more intense light. In other words, only the intensity parameter of the light condition varies from one flash to another that is sent to the eye of the subject, all three other parameters being preferably constant, that is to say a flash of about 600 ms of homogeneous visible white light. In alternative, it would of course be possible to assess the discomfort glare and/or disability glare of the subject in relation with varying spectral and intensity parameters of the light, the two other parameters (spatial and temporal) being constant.

The control unit 15 is furthermore configured to, in step b), record a plurality of neural signals originating from said at least one area of the brain of the subject in response to the exposure of said at least one eye 1 of the subject to each chosen light conditions.

More precisely, the control unit 15 communicates with the neuro-sensor 11 of the device 10 in order for it to detect each neural signal originating from the area of the brain of interest, for instance from the prefrontal area (or from both the prefrontal and the occipital areas of the brain), for each light condition received by the eye 1 of the subject. The memory 150 of the control unit 15 is configured to store each neural signal with the corresponding light condition at which it was recorded.

The processor 151 of the control unit 15 is then configured to compare with each other the recorded neural signals to evaluate a change in brain activity as a function of the light condition received by said at least one eye 1.

The control unit 15 is then configured to determine that the threshold neural signal is the recorded neural signal for which the change in brain activity occurs.

In the following example, it is considered that the discomfort threshold neural signal is reached by the subject at a light intensity smaller than or equal to the light intensity at which the pain threshold neural signal is reached. The disability threshold neural signal is not correlated to the discomfort and pain neural signals. Therefore, the disability threshold neural signal could be reached for a light intensity that is smaller, greater or equal to the light intensity at which are reached respectively the discomfort and pain neural signals.

The change in brain activity is labelled at least by one of the following measures: a lengthening in the time of recovery of the neurons in the neural signals originating from a same area of the brain of the subject, a decrease in the amplitude value of the neural signals originating from a same area of the brain of the subject, at a chosen time after said at least one eye 1 of the subject received the light condition, or a shift in the ratio of maximum amplitude values between the neural signals originating from one area of the brain of the subject and the neural signals originating from another area of the brain activity.

More precisely, in order to compare the signals, the processor 151 is configured to analyze each recorded neural signal and to extract or calculate at least one of the specific features described here-above from said recorded neural signals. For instance, when the recorded neural signal originates from a single area of the brain of the subject, the specific feature could be the time of recovery Tr of the neurons in this area or the amplitude value at a given time. When the recorded neural signals originate from two areas of the brain of the subject, the specific feature could be the ratio between the maximum amplitude values of each recorded neural signal.

The processor 151 is then configured to associate each specific feature to the light condition at which it was obtained. The processor 151 is for instance configured to plot the specific feature as a function of light intensity (see FIGS. 4 and 5), and to determine from these plots, which recorded neural signal is the threshold neural signal, or directly what are the threshold specific features.

On FIG. 4, that represents an example of the time of recovery Tr in milliseconds (ms) of the neurons as a function of light intensity (lux), in a glare conform/discomfort assessment, it can be seen that the curve increases and exhibits two inflection points which indicate two changes in brain activity. The first inflection point is obtained for the light intensity L1 and the second inflection point is obtained for a second light intensity L2.

The processor 151 is configured to determine that the neural signal obtained for the first light intensity L1 is the discomfort threshold neural signal defining the shift from the no-glare state to discomfort glare, and that the neural signal obtained for the second light intensity L2 is the pain threshold neural signal defining the shift from discomfort glare to painful glare. In case none of the neural signals were recorded for the light intensity L1 (respectively L2), the processor 151 is configured to determine that the discomfort threshold neural signal (respectively the pain threshold neural signal) is the recorded neural signal obtained for the light intensity the closest to said first light intensity L1 (respectively L2). In addition, the processor 151 can be configured to determine, for example by reading on the curve, that the discomfort threshold time of recovery that indicates the shift from the no-glare state to discomfort glare is the time corresponding to the first light intensity L1 (for example the discomfort threshold time of recovery is here 300 ms) and that the pain threshold time of recovery that indicates the shift from discomfort glare to painful glare, is the time corresponding to the second light intensity L2 (for example the pain threshold time of recovery is here 600 ms).

The first and second values of light intensity L1, L2 may widely vary from one subject to another, or at least from one group of subjects to another. Indeed, in practice, subjects can be classified into three categories depending on their sensitivity to light. It is estimated that approximately 30% of the population is said “highly sensitive” to light, approximately 50% of the population is a medium sensitive, and approximately 20% of the population if slightly sensitive. The highly sensitive subjects experience discomfort glare and/or disability glare for light conditions that exhibit smaller intensity than the rest of the population, which implies that at least one of the values L1, L2 are smaller for this group of subjects than for the rest of the population. On the contrary, the slightly sensitive subjects experience discomfort glare and/or disability glare for light conditions that exhibit higher intensity than the rest of the population, which implies that at least one of the values L1, L2 are greater for this group of subjects than for the rest of the population.

It appears however that the first and second values of light intensity L1, L2 seem to be independent from the age, the gender, the eye color, the skin color, the pupil size nor the length of the eye of the subjects.

Due to these observations, it is all the more important to be able to determine for each subject, one's personal and individual first and second light intensity values L1, L2, that will lead to individual and personal threshold neural signals.

FIG. 5 represents the maximum amplitude value of the neural signal originating from the prefrontal area (Ap) as a function of light intensity (Lux), and the maximum amplitude value of the neural signal originating from the cortical area (Ac) as a function of light intensity (Lux), in a glare discomfort assessment. On FIG. 5, it can be seen that the maximum amplitude value of the neural signals originating from the prefrontal area (Ap) is constant, while the maximum amplitude value of the neural signals originating from the cortical area (Ac) decreases when the light intensity increases.

The intersection between the two curves Ap and Ac indicates a first change in brain activity, and the point when the two curves are at equidistance from one another indicates a second change in brain activity.

The two curves Ap and Ac intersects for the light intensity L3. The processor 151 is thus configured to determine that the discomfort threshold neural signals defining the shift from the no-glare state to discomfort glare are defined by the respective recorded neural signal originating from the prefrontal and cortical areas obtained for the light intensity L3. The light intensity L3 can be identical to the first light intensity L1 described on FIG. 4, for a same subject.

The two curves Ap and Ac are equidistant from one another from the light intensity L4. The processor 151 is thus configured to determine that the pain threshold neural signals defining the shift from discomfort glare to painful glare are defined by the respective recorded neural signal originating from the prefrontal and cortical areas obtained for the light intensity L4. The light intensity L4 could be identical to the second light intensity L2 described on FIG. 4, for a same subject.

A similar process can be executed in order to determine the disability threshold neural signal defining the shift from no-glare to disability glare, either from only one area of the brain of the subject or from two areas of the brain of the subject. In that case, the subject is asked to execute a visual task, such as try to see a feature (a letter, a word, an image . . . ) while receiving the successive light conditions.

In alternative, the control unit 15 may determine the threshold neural signals with interaction with the subject.

More precisely, the subject is asked to answer a question, for instance to give his feeling or visual ability for each light condition he receives in his eyes. In particular, in order to establish, on one hand, the discomfort threshold neural signal defining the shift from the no-glare state to discomfort glare and, on the other hand, the pain threshold defining the shift from discomfort glare to painful glare, the subject is for example asked to give his feeling between the three followings: comfortable, uncomfortable or painful. In order to establish the disability threshold neural signal defining the shift from the no-glare state to glare disability, the subject is for example asked to give his visual ability between the two followings: able or not able.

The discomfort threshold neural signal is defined as the recorded neural signal that was obtained with the more intense light condition for which the subject still felt comfortable. The pain threshold neural signal is defined as the recorded neural signal that was obtained with the more intense light condition for which the subject felt uncomfortable without feeling painful. The disability threshold neural signal is defined as the recorded neural signal that was obtained with the more intense light condition for which the subject was still able to distinguish a feature in the environment.

Of course, the same process can be executed with more than one subject in order to obtain individual threshold neural signals for each subject and to evaluate a mean threshold neural signals from each individual threshold neural signals. In that case, the processor of the control unit is configured to extract or calculate the specific features of the threshold neural signals established for each subject, and then to calculate mean values of all the extracted or calculated specific features. The mean values thus calculated are chosen as threshold specific features.

For instance, the discomfort threshold neural signal could correspond to:
the recorded neural signal obtained with the light condition of 550 lux or 1000 lux (which is equivalent to a first value of light intensity L1 comprised between 550 and 1000 lux), or to
the recorded neural signal obtained with the light condition of 4000 lux (which is equivalent to a first value of light intensity L1 of approximately 4000 lux), or to
the recorded neural signal obtained with the light condition of 6000 lux (which is equivalent to a first value of light intensity L1 of approximately 6000 lux).

When it appears that several subjects obtain the discomfort threshold neural signals for the same range of intensities, then the results obtained for these subjects could be gathered together to evaluate a mean threshold neural signal. In the present example, the subjects for which the discomfort threshold neural signal is obtained for a light condition comprised between 550 lux and 1000 lux, could form a “highly sensitive group of people”, the subjects for which the discomfort threshold neural signal is obtained for a light condition of 4000 lux could form a “medium sensitive group of people,” and the subjects for which the discomfort threshold neural signal is obtained for a light condition of 6000 lux could form a “slightly sensitive group of people”.

In an alternative embodiment of the device (not shown), the neuro-sensor may be able to detect the neural signal that originates from the retina of the subject. Such neural signals are called electroretinograms.

The neural signal detected by such electrodes should exclude all the electrical activity that could be induced by muscular activity of the eye, that would for instance be linked to an eyelid movement. In other words, the neural signal of interest in the present invention focuses only on the sensory information sent to the brain of the subject, through the optic nerve, by the photoreceptor of the eye(s) receiving the light condition. The electrical activity that is due to muscular activity of the eyes is called a “muscular signal”. The muscular signal possibly mixed with the neural signal is considered to be a noise detected by the electrodes and should be removed from the neural signal. In practice, the muscular signal generally exhibits an electrical activity of several millivolts (mV), whereas the neural signal on which the invention focuses exhibits an electrical activity of a few microvolts (μV or micro V) only. Moreover, in the frequency domain, the frequencies exhibited by a muscular signal are generally comprised between 0.1 and 0.5 Hertz (Hz), whereas the frequencies exhibited by a neural signal according to the invention are generally around 5 to 10 Hz. Such distinctive features help removing the muscular signal from the neural signal, in cases both are detected by the electrodes.

In order to record such neural signal originating from the retina of the subject, the electrodes are placed around the eye of the subject. Notably, the electrodes are located on the eyelid located below the eye, and/or at the external corner of the eyes (that is to say at the external intersection of the eyelids located under and above the eye). One reference electrode is also located on the temple of the subject or in the center of the forehead.

In a further aspect of the invention, the device 10 is embedded in a virtual reality headset 20; 40 intended to be worn by a subject, as shown on FIGS. 2A and 2B.

More precisely, the virtual reality headset 20; 40 may comprise:
the device 10 as described above; and
a fastening unit 21; 41 for keeping said device 10 in front of the eyes 1 of the subject.

Such virtual reality headset 20; 40 is an optoelectronic device, i.e. an electronic device that source, detect and/or control light.

As shown on FIGS. 2A and 2B, the virtual reality headset 20; 40 is configured to face both eyes of the subject when it is worn by the subject. Particularly, the headset 20; 40 is a binocular device so that it is configured to face each eye of the subject when it is worn by the subject. Alternatively, the headset 20; 40 may be monocular and configured to face only one eye area of the subject.

Dimensions and weight of the headset 20; 40 are configured to make it possible for the subject to handle it in front of its eyes using the fastening unit 21; 41. The fastening unit 21; 41 represented on the embodiments of FIGS. 2A and 2B comprises means for fastening the headset 20; 40 to the user's head such as straps able to surround the subject's head or such as spectacle arms positioned onto the subject's ears.

Alternatively, the fastening unit may comprise a support leg configured to sit on a table or on the ground.

Alternatively, the fastening unit may comprise handles for the subject to place its hands and position the headset 20; 40 in front of its eyes, as the subject would do with binoculars.

As shown on FIGS. 2A and 2B, the electrodes 110 of the neuro-sensor 11 of the first and second embodiments of the headset 20; 40 are placed on strips located in the headset 20; 40 in order to touch the appropriate region of the face or of the head of the subject when the headset 20; 40 is worn. More precisely, in the first embodiment of the headset 20 represented on FIG. 2A, a first series of electrodes 110 are placed above a cavity 16 formed by a casing 23 of the headset 20 intended to house the light source 12, and a second series of electrodes are placed along the straps of the fastening unit 21. Such first embodiment of the headset 20 is particularly well suited to detect the neural signal linked to the sensitivity of the eye that originates from the brain of the subject and more particularly from the prefrontal and cortical areas of the brain. In the second embodiment of the headset 40 represented on FIG. 2B, a first series of electrodes 110 are placed under a cavity 16 formed by a casing 43 of the headset 40 intended to house the light source 12, and a reference electrode 110ref is placed above said casing 16, so as to be located on a vertical line passing in between the eyes and aligned along the nose of the subject. The reference electrode 110ref is therefore located in the center of the forehead of the subject when the headset 40 is worn by said subject. Such second embodiment of the headset 40 is particularly well suited to detect the neural signal linked to the sensitivity of the eye that originates from the retina of the subject.

As shown on FIGS. 2A and 2B, the headset 20; 40 may comprise the at least one light source 12 for stimulating at least one eye 1 of the subject with the light condition. Said light source 12 is preferably located in the cavity 16 formed by the casing 23 of the headset 20; 40. The diffuser is disposed in front of the light source 12 and closes the cavity 16. When the headset 20; 40 is worn by the subject the diffuser faces the eyes 1 of the subject and is located in between the light source 12 and said eyes 1.

The virtual reality headset 20; 40 may comprise an isolating unit 22; 42 for isolating the subject from ambient light.

The isolating unit 22; 42 may comprise a black foam adapted to follow the curve of the face of the subject and to block any light coming from outside the cavity 16 of the headset 20; 40 to enter the eye 1 of the subject.

The virtual reality headset 20; 40 may further comprise holding means (not represented) for holding a light filter in front of the eyes of the subject. Such holding means may for instance be a groove or a slot adapted to receive a part of the edge of the filter. Such groove could for instance border the cavity 16 of the headset 20; 40. The light filter would therefore be placed in between the eyes 1 of the subject and the light source 12. In that way, the headset 20; 40 may be used to test the effect of a light filter on the glare state of the subject.

The control unit 15 may be fully or partly embedded within the headset 20; 40. When it is partly embedded only, the unit control 15 is disposed within an external terminal.

Furthermore, the device 10 comprises an accumulator (not represented) to be self-sufficient in energy.

In a further aspect of the invention, the device 10 is embedded in an eyeglass 50 intended to be worn by a subject, as shown on FIGS. 6A et 6B.

More precisely, such eyeglass 50 comprises:
the device 10 of the invention, and
an active light filter defined at least by one variable parameter chosen among the transmission value and/or the spectrum range.

The active light filter is embedded on the glass 51 of the eyeglass 50. The electrodes 110 of the device are deployed from the eyeglass 50 in order to extend in the areas of the head that will give access to the neural signal that originates from the prefrontal area of the brain of the subject and eventually to the neural signal that originates from the cortical area of the brain of the subject. The device 10 is for example embedded in the frame 52 of the eyeglass 50, or in the vicinity of the portion of frame 52 that ends behind the ear of the subject.

Here the light filter should be able to modify the light condition before it reaches the eye of the subject in order to prevent the subject from experiencing discomfort glare and/or disability glare anymore, although in the absence of said light filter the subject experiences discomfort glare and/or disability glare. The light filter is able to modulate (or modify) at least one of the parameters (among the spectral, spatial, temporal and intensity parameters) of the light condition that passes through it.

In the present example, the light filter is considered to be an optical system that reduces and/or filters the light intensity (intensity parameter of the light condition) and/or that modifies the range of wavelength (spectral parameter) of the light condition that passes through it. As such, the light filter is here described by its transmission value (Tv) that indicates the intensity of light that can get through the filter (the rest of the light being blocked by said light filter), and/or by its spectrum range that indicates the wavelengths (or ranges of wavelengths) that can get though said light filter (the other wavelengths being blocked by said filter).

In practice, the transmission value Tv is equivalent to the luminous transmittance of the filter, which is a ratio of the luminous flux transmitted by the filter to the incident luminous flux. In other words, the luminous transmittance defines the percentage of light from a light flux transmitted through the filter.

Therefore, the greater the transmission value (or luminous transmittance), the greater the light intensity of the light condition that can reach the eye of the subject through the filter. A surface with a luminous transmittance of 0% prevents the whole light flux to pass through the surface whereas a surface with a luminous transmittance of 100% allows the whole light flux to pass through it without absorbing it.

The luminous transmittance in the visible spectrum may be determined using the equation as follows:

$$Tv = 100 \times \frac{{\int }_{380 nm}^{780 nm} T ( λ ) \cdot V ( λ ) {S}_{D 65 λ} ( λ ) \cdot d λ}{{\int }_{380 nm}^{780 nm} V ( λ ) \cdot {S}_{D 65 λ} ( λ ) \cdot d λ} \%$$

where

T(λ) is the spectral transmittance of the filter;

V(λ) is the spectral luminous efficiency function for daylight (see ISO/CIE

S_{D65λ}(λ) is the spectral distribution of radiation of the illuminant D65 according to the standard of the International Commission on illumination (see ISO/CIE 10526).

The light filter is here considered to be an active filter such as photochromic or electrochromic filters. It means that the transmission value and/or the spectrum range of the light filter can change over time, depending on the real light condition that the subject who is wearing the eyeglass is experiencing. As such, said parameter defining the filter (transmission value and/or spectrum range) are “variable”. The real light condition means here that the light condition is the one that is naturally surrounding the eyeglass. It could for instance be an indoor and artificial light condition, a punctual light condition in the night, a bright and wide light condition on a sunny day on the ice or on the sea.

The control unit 15 of the device 10 embedded on the eyeglass 50 of the invention is adapted to determine said variable parameter of the active filter based on the assessing of the discomfort glare and/or disability glare implemented by said device 10 when the subject receives the real light condition through the glasses of said eyeglass 50.

For instance, the eye of the subject receives a given light condition, trough the eyeglass 50, the electrodes 110 detect the neural signal(s) in the prefrontal and eventually in the cortical area of the brain of the subject, said neural signal(s) being then analyzed by the control unit 15 in order to assess whether said given light condition led to a discomfort glare and/or disability glare of the subject. If the conclusion of the control unit 15 is that the subject is experiencing a discomfort glare and/or disability glare due to said given light condition, then the control unit 15 modifies the transmission value and/or spectrum range of the filter in order for the subject to be in no-glare state under the same real light condition. In other words, the real light condition has not changed, but the light condition received by the eye of the subject through the eyeglass 50 has changed thanks to the active filter.

In a preferred embodiment, the parameters of the active filter are set depending on the real light condition surrounding the subject wearing the eyeglass 50, in order to for the subject to be as close as possible from a discomfort glare and/or disability glare, without experiencing said glare.

As an alternative, the eyeglass of the invention comprises:
a sensor to detect at least one of the parameters of the light conditions,
an active filter defined at least by one variable parameter chosen among the transmission value and/or the spectrum, and
a control unit able to determine the variable parameter of the active filter based on said parameter of the light condition and on the light condition under which is obtained the threshold neural signal NS₀ that is characteristic of a shift for the subject from no-glare state to discomfort glare and/or disability glare.

Process

The invention also relates to a method for objectively assessing discomfort glare and/or disability glare of a subject, comprising the following steps:

a) recording the neural signal linked to the sensitivity of the eyes in at least one area of the brain of the subject while at least one eye of the subject receives a given light condition,

b) providing a threshold neural signal that is characteristic of a shift for the subject from a no-glare state to discomfort glare and/or disability glare,

c) assessing whether the neural signal recorded in step a) correlates with a discomfort glare and/or disability glare of the subject by comparing said neural signal recorded in step a) to said threshold neural signal provided in step b).

The device 10 is able to implement the method. The method was fully described in relation to the device 10.

In step a) of the method, the subject is silent and still, in a silent environment. Therefore, the recorded neural silent exhibits mainly the response of the neurons to the light stimulation, and not to any other possible stimulation.

In a preferred embodiment, the subject is seated rather than standing on its feet in order to increase the stillness.

For the assessment of glare discomfort, the method only stimulates the eye 1 of the subject with a light condition in step a). For the assessment of glare disability, the method both stimulates the eye 1 of the subject with a light condition and shows the eye a feature that the subject tries to see, in said step a). For instance, for the assessment of disability glare, the subject may try to read a letter, a word or a sentence, or may be asked to compare two images, while receiving the successive light conditions.

In step a), the method may further comprise a step of measuring the diameter of the pupil of the subject in order to set up the intensity parameter of the light condition to be sent to the eye of the subject, inTroland.

The present invention is not limited to the embodiments described and shown, but the skilled person can make any modifications according to the invention.

Notably, in addition, or in replacement of the threshold neural signals, the memory of the control unit could be configured to store the threshold specific features, that could be, as explained above, personal or mean specific features.

Other specific features that the one described here-above (time of recovery, amplitudes, ratio of activity from one area to another) can be used to compare the neural signals.

Neural signals originating from other areas of the brain than the prefrontal and cortical areas can be detected and analyzed. The more neural signals are detected and analyzed, the more precise is the determination of the state of subject under the given light condition.

According to another embodiment, the control unit 15 may be adapted to further provide a machine learning algorithm. Such machine learning algorithm may notably be used in order to provide the threshold neural signal NS0 in step b).

A machine learning algorithm takes as input a training set of observed data points to “learn” a data structure such as an equation, a set of rules, or some other data structure. This learned data structure or statistical model may then be used to make generalizations about the training set or predictions about new data. As used herein, “statistical model” refers to any learned and/or statistical data structure that establishes or predicts a relationship between two or more data parameters (e.g., inputs and outputs). Although the invention is described below with reference to neural networks, other types of statistical models may be employed in accordance with the present invention. For example, each data point of the training data set may include a set of values that correlate with, or predict, another value in the data point.

In the present invention, the machine learning algorithm may be configured to correlate the recorded neural signals provided to the machine learning algorithm (input) to a threshold neural signal NS0 that is characteristic of a shift for the subject from no-glare state to discomfort glare and/or disability glare (output). In other words, the input of the machine learning algorithm may be the recorded neural signals and the output may be a threshold neural signal NS0 that is characteristic of a shift for the subject from no-glare state to discomfort glare and/or disability glare.

The embodiment using the machine learning algorithm to determine the threshold neural signal NS0 may be taken alone or in combination with the other embodiments explained here above to determine the threshold neural signal NS0.

Said machine learning algorithm of the control unit 15 may be based either on a Long short-term memory (LSTM) technique or a convolutive neural network (CNN).

LSTM technique is part of recurrent neural networks (RNNs). Classical RNNs techniques comprise a network of neural nodes organized in successive layers. Each node (also called neuron) in a given layer is connected one-way to each of the nodes in the next layer. This structure allows previous moments to be taken into account in the neural network, since a first layer for a former moment t−1 is connected to a second layer for a current moment t. This second layer is also connected to a third layer for a subsequent moment t+1, and so on with a plurality of layers. Each signal provided as an input is therefore processed in a temporal way, taking into account the signals provided at former moments.

CNN techniques use the signals as images, not in a temporal way. The plurality of acquired signals is processed at once with all the data acquired during a given test duration. Mathematical image processing operations are then applied to the image obtained with the plurality of acquired signals, e.g. convolution integral, to determine outputs of the machine learning algorithm.

The machine learning algorithm may comprise a guiding model defining determination rules, said guiding model being configured to guide the prediction of the machine learning algorithm. These rules may comprise sub-correlations between recorded neural signals and a threshold neural signal NS0. For example, this guiding model may provide that a given variation of a characteristic has to be correlated to a certain threshold neural signal. In another example, the guiding model may provide that a predetermined combination of variation of characteristics implies a threshold neural signal or a list of threshold neural signal. This guiding model allows easing the correlation made by the machine learning and therefore both reduces the time taken by the correlation and improves its accuracy.

The control unit 15 may use a machine learning algorithm which is already trained, i.e. the neural network of the machine learning algorithm already comprises an equation or a set of rules configured to provide a correlation between a recorded neural signal and a threshold neural signal. Alternatively, the control unit 15 is configured to train the machine algorithm to determine the correlation.

Training of the machine learning algorithm is preferably performed by providing the algorithm with a plurality of recorded neural signals related to a set of initial users. By “initial users” we mean users which participate to the learning of the machine learning algorithm. In other words, initial users provide data allowing the machine learning algorithm to correlate recorded neural signals to threshold neural signal. On the contrary, a “target user” refers to a user for which a behavior determination is performed on the basis of the machine learning algorithm, i.e. for which a prediction of his behavior may be performed.

This training is repeated many times to make the algorithm more accurate. As an example, training the algorithm may imply at least one hundred initial users.

A machine learning algorithm is able to process very complex signals and then correlate said complex signals to certain behaviors.

The invention also relates to a method for determining a parameter that is characteristic of a light filter to be provided to a subject in order to maintain or improve the visual comfort and/or visual acuity of said subject, when a given light condition is sent to the eye of the subject.

As explained hereabove, the light filter should be able to modify the light condition before it reaches the eye of the subject in order to prevent the subject from experiencing discomfort glare and/or disability glare, although in the absence of said filter the subject would otherwise experience discomfort glare and/or disability glare. The goal of the method of the invention is even to be able to provide the subject with a light filter that will avoid him to experiment discomfort and/or disability glare due to any of the light conditions that he may come across in his life, or at least that he may come across in his daily life.

For instance, highly sensitive people will be disturbed by all kind of light condition, both indoor and outdoor, artificial, and natural light. They therefore need a light filter whatever the light condition they receive in their eyes. On the contrary, slightly sensitive people will express discomfort only for very bright light condition. They will therefore need a light filter only when they receive a light condition with a high intensity, such as the light condition of outdoor environments.

The light filter is able to modulate (or modify) at least one the parameters of the light conditions that passes through it, among the spectral, spatial, temporal and intensity parameters of said light condition.

In the present example, the light filter is considered to be an optical system that reduces and/or filters the light intensity (intensity parameter of the light condition). As such, the light filter is here described by its transmission value (Tv) that indicates the intensity of light that can get through the filter (the rest of the light being blocked), as defined hereabove.

Here, the light filter may be a filter coating or a filtering function which can be used to provide a filter coating. The filter may be in the form of a passive filter (uniform, with a gradient or with a spatial variation), for instance a tinted glass, or an active filter such as photochromic or electrochromic system.

More precisely, the method of the invention comprises the following steps:

determining a threshold light condition characteristic of a shift for the subject from no-glare state to discomfort glare and/or disability glare,

determining for each light condition among a group of light conditions, an index representative of the level of protection required by the subject, based on said light condition threshold,

determining a score for each light condition among the group of light conditions and for each filter among a group of filters, said score being representative of the capacity of the filter to reach the level of protection required by the subject, based on said index, and

determining at least one filter among the group of filters based on the scores of said at least one filter in a plurality of light conditions among the group of light conditions.

The first step of the method of the invention is to determine the threshold light condition that is characteristic of a shift from no-glare state to discomfort and/or disability state for the subject. Such determination of the threshold light condition is here implemented by the device of the invention described hereabove and more precisely by the device according to the embodiment wherein the assessing of the glare of the subject is based on the analysis of the comparative activity of the neurons in two areas of the brain of the subject.

More precisely, as explained previously, to do so, the processor 151 of the control unit 15 of the device 10 of the invention operates the light source 12 to expose said at least one eye of the subject to a plurality of given light conditions at a plurality of chosen times.

In the present example and for the sake of simplicity, we will consider that the successive light conditions provided to the eye of the subject only varies as regards their respective intensity parameters, and are constant over their other parameters (temporal, spectral and spatial parameters). However, of course, other parameters of the light condition could be tested, preferably one at a time.

Each corresponding neural signal recorded for each light condition is then stored in the memory 150 of the control unit 15.

The control unit 15 then determines that the threshold light condition is the light condition for which the ratio of maximum amplitude values between the neural signals NS originating from said at least one area of the brain of the subject and the neural signals NS originating from another area of the brain activity is the closest to a threshold ratio characteristic of a shift for the subject from no-glare state to discomfort glare and/or disability glare.

When the threshold ratio is known ahead of this method of the invention, the control units determines that the threshold light condition is the light condition that led to the recorded neural signals which ratio is the closes to said known threshold ratio. For instance, in the example described above, the discomfort threshold ratio is comprised between 0.8 and 1.2. Let us say that said discomfort threshold ratio is here equal to 1. The control units thus determines that the threshold light condition is the light condition that led to recorded neural signals which ratio is the closest to 1.

Alternatively, when the discomfort threshold ratio is not known ahead of this method of the invention but determined thanks to the device of the invention described previously, the processor 151 can determine that the threshold light condition is the light intensity L1, which is the light intensity associated with the discomfort threshold neural signal (see FIG. 4).

The next step comprises the determination of a subject index for each light condition, among various light conditions to which the subject can be exposed, ether in his whole life or in his daily life.

The subject index represents the level of protection required by the subject, for each light condition.

By “level of protection required by the subject”, it is meant a level of protection based on answers or inputs coming from the subject himself, via a questionnaire. Hence, the purpose of this step is to determine:
the subject's need of protection (anamnesis), and
which kind of light condition does the subject face and for which he needs protection.

This step allows determining, and potentially selecting, the light conditions from which the best filter will be chosen.

The light conditions are frequent daily situations that can be a source of discomfort for the subject. The group of light conditions is selected among a set of conditions wherein each light condition is defined, as described before, by the intensity, spectral, temporal and spatial parameters. The conditions of the set are preferably associated with different combinations of said parameters.

According to a preferred embodiment, each light condition is selected to depict a specific combination of the four light parameter (intensity, spatial, spectral and temporal parameter). Hence, the group as a whole is determined to have the most representative parameters of the 4 light parameters gathered in different light conditions. For instance, a night situation may imply medium to high light intensity (intensity parameter) with movable light sources (spatial parameter) which may be only emitted toward the subject during a few seconds (temporal parameter). The group of light conditions preferably comprises at least one outdoor situation, at least one indoor situation and at least one night situation.

The subject index is determined for a given light condition among the group of light conditions by a calculation based on the intensity parameter of the light condition and on the determined threshold light condition determined in the previous step. Particularly, the subject index is preferably calculated as the ratio between the intensity parameter associated with said light condition and the light intensity of the threshold light condition.

$$Subject user = \frac{Intensity parameter}{Threshold light intensity}$$

For instance, when a threshold light intensity of 600 Lux is determined in the previous step, the subject index is equal to 10 for a light condition having a light intensity parameter of 6000 Lux. It means that the light condition is ten (10) times more intensive than what the subject is able to find comfortable (and tolerate).

A subject index is determined for each intensity of light condition, preferably for each selected light condition.

Then, in a further step, the score associated with each filter and each light condition is determined. More precisely, the score determining step comprises a sub-step of determining an index representative of the protection provided by a given filter, called the filter index.

The filter index defines the amount of light that the filter is able to cut. The filter index is determined using the equation as follows:

$$Filter index = \frac{100}{Tv}$$

A filter may have a single luminous transmittance value, i.e. a fixed value, or a plurality of luminous transmittance values, as a photochromic or electrochromic lens. When the filter is a varying filter, the lower and higher luminous transmittance values are preferably calculated to determine the compliance of the filter to the user for each lower and higher luminous transmittance values.

The score determining step further comprises a sub-step of calculation of the score for each light condition among the group of light conditions and for each filter among a group of filters. Said score is representative of the effectiveness of each filter in a given situation, i.e. the ability of each given filter to reach the level of protection required by the subject.

The score for a given filter and a given light environment 40 is calculated based on the subject index in said given light condition and the filter index of said given filter. Particularly, the score is calculated as the ratio between the subject index and the filter for said given filter and said given light condition. For instance, the score is equal to 1 for a filter index of 6.67 and a user index of 6.67. A score of 1 means that the filter covers 100% of the subject's needs of protection for said light condition. If a filter index is higher than 1, it means that the filter fully protects the subject so that light comfort is optimal but there is a risk of vision loss (loss of visual acuity).

The score is therefore a score representative of the compliance for a subject of a given filter in a given light condition. Providing the score for selected light condition which have been identified by the subject as having a high level of discomfort and/or recurrence allows to help determining the best protection for the subject.

Finally, it is then possible to determine at least one filter among the group of filters based on the scores determined for each filter and each light condition among the group of light conditions.

This step aims at ranking the filters based on their scores for the different light conditions. Preferably, the filters are ranked only with regard to the light conditions selected by the subject, that is to say for the light conditions of the group of light conditions that is the subject is likely to experience while wearing the filter.

Each score is associated with a value representative of the compliance of a given filter in a given light condition in view of the level of protection required by the subject. Then, a global value may be determined based on all the values determined for a same filter. All the values of a same filter for each light condition or each selected light condition may be added to obtain this global value. The plurality of filters is then ranked based on the global values.

According to a preferred embodiment, one or more filters are determined for at least two transparent supports having different purposes. For example, when the transparent support is an ophthalmic lens, one or more filters are determined for at least two spectacles having different use. One spectacle may be used as sunglasses and another spectacle may be used as everyday eyeglasses. The ranking of the filters is performed with regard to specific light environment which are associated to the use of the transparent support.

According to a preferred embodiment, the closer to 100% the score is, the higher the value is. Particularly, scores from a low compliance threshold to a high compliance threshold may be associated to a positive value whereas scores out of this compliance range may be associated to a negative value. In doing so, the global value of each filter is weighted based on a predetermined degree of compliance to the subject's need. For instance, the compliance range may be set from 86% to 200%. Furthermore, scores which are considered to be significantly non-compliant to the user's need, e.g. scores under 50% and above 300%, may be associated to a low value. As an example, scores between 86% and 200% are associated with a value of 2, scores lower than 50% or high than 300% are associated with a value of −2 and scores from 51% to 85% or from 201% to 299% are associated with a value of −0,5. Therefore, if we consider a filter having scores equal to 42, 104, 174, 123 and 185, the respective associated values would be −2, 2, 2, 2 and 2. The global value, i.e. the sum of the values, would be 6. The scores which are summed are at least those which have been selected in the step of determination of the subject index.

One or more filters may be then determined to have the best compliance with the subject's protection need. Preferably, at least two filters from different categories of filters are determined to provide the user or the ECP with a broader list of compliant filters. Preferably, these different filter categories correspond to different purposes or for different pair of spectacles. Different categories of filter may be filters intended to be put on sunglasses and filters intended to be put on everyday eyeglasses.

This method of the invention may be a computer-implemented method which can be performed using code instructions from a computer program product or a computer system. The computer system comprises a processor; and a memory with computer code instructions stored thereon. The memory operatively is coupled to the processor such that, when executed by the processor, the computer code instructions cause the computer system to perform the filter determining method.

As explained previously, the filter can in addition be described by other parameters (rather than only the transmission value Tv), notably:
its spectrum range, and,
for photochromic or electrochromic filters, its dynamic law of variation of the Transmission value (Tv) (for the electrochromic filter) of its dynamic law of variation of the spectrum range (for a photochromic filter).

Ideally, the choice of the spectrum range and the dynamic laws of variation of the photochromic and/or electrochromic filter can be selected with the same approach as described hereabove in relation with the transmission value Tv, so as to select the right parameter to optimize the neural processes, without letting the subject experiencing discomfort and/or disability glare.

A combination of these parameters (between Transmission value Tv, Spectrum range, or dynamic laws of variation) could be interesting to manage the selection of the best combination of parameters for the filter to optimize the comfort and visual acuity.

According to an embodiment of this method of the invention, the control unit determines a law of variation of the transmission value of the filter and/or the law of variation of the spectrum range based on the light condition received by said filter.

Such embodiment of the method is implemented with the device of the invention. Different options are available to test the effect of each transmission value and/or spectrum range on the neural process of the subject: either the subject physically wears a filter of given transmission value (or of given spectrum range) in front of his eyes so that the light received by said eye of the subject first goes through the given filter, and/or the light condition sent to the eye of the subject is artificially modified as if it passed through a filter (of given spectrum range and/or of given transmission value) in order to imitate the effect of said filter, without the subject needing to physically wear the filter.

The dynamic law of variation of the transmission value is determined as follows: a plurality of light conditions of different intensity are successively sent to the subject, and for each light condition sent to the eye of the subject that leads to a ratio (of maximum amplitude values between the neural signals originating from one area of the brain of the subject and the neural signals originating from another area of the brain of the subject) strictly greater than the threshold ratio, the transmission value Tv of the filter is decreased in order to force said ratio to be equal to said threshold ratio.

In other words, when the ratio is greater than the threshold ratio, the subject needs a protection, or a filter with a lower transmission value (or a different spectrum range) than the one tested. On the contrary, when the ratio is smaller or equal to said threshold ratio, the subject has the appropriate filter for the tested light condition.

The law of variation aims at analyzing how the light intensity impacts said ratio of maximum amplitude values and how the transmission value of the filter should therefore evolve to prevent the subject from experiencing discomfort glare and/or disability glare.

The law of variation therefore obtained is used for configuration of an electrochromic system that exhibits a variable transmission value.

In alternative, the law of variation is used to select the fixed tint of a filter, said filter being also selected depending on the light conditions wherein the subject is the most likely to wear said filter.

In alternative or in complement, the dynamic law of variation of the spectrum range is determined very similarly as the law of variation of the transmission value except that the ratio is obtained for a plurality of light conditions of different spectral parameter and that the spectrum range of the filter is modified depending on the comparison between the ratio and the threshold ratio for the subject not to experience discomfort glare nor disability glare.

1. A device for assessing discomfort glare and/or disability glare of a subject, comprising:
at least one neuro-sensor for detecting a neural signal linked to the sensitivity of the eyes of the subject,
a control unit adapted to
a) record the neural signal of the subject detected by the neuro-sensor while at least one eye of the subject receives a given light condition,
b) provide a threshold neural signal that is characteristic of a shift for the subject from no-glare state to discomfort glare and/or disability glare,
c) assess whether the neural signal recorded in step a) correlates with a discomfort glare and/or disability glare of the subject by comparing said neural signal recorded in step a) to said threshold neural signal provided in step b).

2. The device of claim 1, wherein the neural signal originates from at least one specific area of the brain.

3. The device of claim 2, wherein in step c), the control unit is adapted to:
determine at least one specific feature of the recorded neural signal originating from said at least one specific area of the brain of the subject,
compare said at least one specific feature of the recorded neural signal to a threshold specific feature of the threshold neural signal originating from the same at least one specific area of the brain of the subject, and
depending on the result of said comparison, assess whether the subject is experiencing discomfort glare and/or disability glare under said given light condition.

4. The device of claim 3, wherein the specific feature of the neural signal is at least one of the specific features chosen from the following list: a time of recovery of the stimulated neurons in said at least one specific area of the brain of the subject, or an amplitude value of the neural signal originating from said at least one area of the brain of the subject at a chosen time after said at least one eye of the subject received said given light condition.

5. The device of claim 1, wherein the neuro-sensor is further able to detect the neural signal originating from another area of the brain of the subject.

6. The device of claim 1 wherein in step c), the specific feature of the neural signals that is determined by the control unit is the ratio between a maximum amplitude value of the neural signal originating from said at least one area of the brain and a maximum amplitude value of the neural signal originating from said other area of the brain.

7. The device of any of claim 1, further comprising at least one light source that is operated by the control unit to expose said at least one eye of the subject to said given light condition.

8. The device of claim 7, wherein the control unit is adapted to operate said light source to expose said at least one eye of the subject to a plurality of given light conditions at a plurality of chosen times, and wherein in step b), the control unit is furthermore adapted to:
record a plurality of neural signals originating from said at least one area of the brain of the subject in response to the exposure of said at least one eye of the subject to each chosen light conditions, and,
compare with each other the recorded neural signals to evaluate a change in brain activity as a function of the light condition received by said at least one eye,
determine the threshold neural signal as the recorded neural signal for which the change in brain activity occurs.

9. The device of claim 8, wherein the change in brain activity is labelled at least by one of the following measures: a lengthening in the time of recovery of the neurons in the neural signals originating from a same area of the brain of the subject, a decrease in the amplitude value of the neural signals originating from a same area of the brain of the subject at a chosen time after said at least one eye of the subject received the light condition, or a shift in the ratio of maximum amplitude values between the neural signals originating from said at least one area of the brain of the subject and the neural signals originating from another area of the brain activity.

10. The device of any of claim 1, wherein there is a discomfort threshold neural signal defining the shift from the no-glare state to discomfort glare and a pain threshold neural signal defining the shift from discomfort glare to painful glare and a disability threshold neural signal defining the shift from the no-glare state to disability glare.

11. The device according to claim 1, wherein in step a), the eye of the subject is provided with a filter through which the light passes before reaching the at least one eye of the subject.

12. The eyeglass comprising:
the device as claimed in claim 1, and
an active filter defined at least by one variable parameter chosen among a transmission value and/or a spectrum range,
wherein the control unit is adapted to determine a value for said variable parameter of the active filter based on the assessing of the discomfort glare and/or disability glare implemented by said device when the subject receives the light condition through said eyeglass.

13. The method for assessing discomfort glare and/or disability glare of a subject, comprising the following steps:
a) recording a neural signal that originates from at least one specific area of the brain of the subject while at least one eye of the subject receives a given light condition,
b) providing a threshold neural signal that is characteristic of a shift for the subject from no-glare state to discomfort glare and/or disability glare,
c) assessing whether the neural signal recorded in step a) correlates with a discomfort glare and/or disability glare of the subject by comparing said neural signal recorded in step a) to said threshold neural signal provided in step b).

14. A virtual reality headset intended to be worn by a subject, comprising:
a device as claimed in claim 1; and,
a fastening unit for keeping said device in front of the eyes of said subject.

15. A method for determining a parameter that is characteristic of a light filter to be provided to a subject in order to maintain or improve the visual comfort and/or visual acuity of said subject for a given light condition, comprising the following steps:
determining a threshold light condition characteristic of a shift for the subject from no-glare state to discomfort glare and/or disability glare,
determining for each light condition among a group of light conditions, an index representative of the level of protection required by the subject, based on said light condition threshold,
determining a score for each light condition among the group of light conditions and for each filter among a group of filters, said score being representative of the capacity of the filter to reach the level of protection required by the subject, based on said index, and
determining at least one filter among the group of filters based on the scores of said at least one filter in a plurality of light conditions among the group of light conditions,
wherein the determination of the threshold light condition is implemented by the device of claim 7 wherein the control unit:
operates said light source to expose said at least one eye of the subject to a plurality of given light conditions at a plurality of chosen times, and
determines that the threshold light condition is the light condition for which the ratio of maximum amplitude values between the neural signals originating from said at least one area of the brain of the subject and the neural signals originating from another area of the brain activity is the closest to a threshold ratio characteristic of a shift for the subject from no-glare state to discomfort glare and/or disability glare.
